(19) United States
(12) Patent Application Publication
(10) Pub. No.: US 2026/0174938 A1
(43) Pub. Date: Jun. 25, 2026

(54) INDWELLING MEDICAL DEVICE

(71) Applicant: ACatechol, Inc., Santa Barbara, CA (US)

(72) Inventors: Roscoe T. Linstadt, Palo Alto, CA (US); Kollbe Ando Ahn, Las Vegas, NV (US)

(21) Appl. No.: 19/540,347

(22) Filed: Feb. 13, 2026

Related U.S. Application Data
(63) Continuation of application No. PCT/US2024/042818, filed on Aug. 16, 2024.
(60) Provisional application No. 63/533,236, filed on Aug. 17, 2023.

(51) Int. Cl. A61L 29/08; A61L 29/06; A61L 29/16

(52) U.S. Cl. CPC A61L 29/085; A61L 29/06; A61L 29/16; A61L 2300/406

(57) ABSTRACT

In examples, the present application discloses an indwelling medical device such as a catheter and other medical devices, which may include a core region and a surface region which may include an interpenetrated polymer network, the interpenetrated polymer network may include a primary polymer network and a secondary polymer network.

### RELATED APPLICATIONS

This application is a continuation of International Application No. PCT/US2024/042818 filed Aug. 16, 2024, which claims priority to U.S. Provisional Patent Application No. 63/533,236, filed Aug. 17, 2023, each of which is hereby incorporated by reference in its entirety.

### BACKGROUND

### Technical Field

The present application relates to anti-fouling and anti-infective polymeric surfaces, surface coatings, and method of use.

### Description of the Related Art

Within the field of anti-fouling and anti-infective polymeric surfaces, it is established that zwitterions can be grafted onto surfaces as coatings to confer resistance against biofouling or biofilm formation, commonly referred to as “foul-release” coatings. Surfaces adorned with zwitterionic polymers possess the remarkable ability to repel biomolecules and thwart their adhesion to biomedical surfaces within the human body.

However, despite zwitterions' renowned foul-release attributes, coupled with their exceptional biocompatibility and hemocompatibility, they have not been extensively employed in practical anti-infective surface applications. The limited usage of zwitterionic coatings in real-world scenarios can be attributed to two primary reasons. First, zwitterionic polymers exhibit superhydrophilicity, making existing coatings susceptible to inadequate durability in physiological environments. Second, zwitterionic surfaces do not provide antimicrobial activities.

Accordingly, there exists a need for hydrophilic coatings including zwitterionic coatings that possess the prolonged antimicrobial activities that reduce pathogenic biofilm formation on indwelling medical devices. Previously, catheters have been coated with zwitterionic polymers. Smith et al. Sci Transi Med. 2012 Sep. 26; 4(153):153ra132 (hereinafter “Semprus”), which is hereby incorporated by reference, teaches surface grafting using a zwitterionic polymer, providing a surface modification. However, Semprus does not utilize an interpenetrated polymer network. Additionally, catheters have been impregnated with antibiotics. Commercially available antimicrobial Cook Spectrum CVC (hereinafter “Cook Spectrum”) contains rifampin and minocycline hydrochloride. However, Cook Spectrum does not repel and prevent the adhesion of biomolecules to the CVC.

US 2011/0282005A1, which is hereby incorporated by reference, teaches that an IPN can be formed between a zwitterionic polymer network and a segmented polyurethane (SPU) network to prevent biofouling. However, US 2011/0282005A1 does not describe the specific portion or thickness of the IPN relative to the pristine SPU substrate. Instead, it discloses that the surface area has a relatively high zwitterionic content (zwitterion-rich), while the core area contains a relatively high PU content (PU-rich).

Further, US 2011/0282005A1 does not disclose testing of the mechanical properties (such as tensile strength, hardness, elongation, modulus) of the final SPU material, where the IPN portion was over 40%.

### SUMMARY

In some examples, disclosed herein are medical devices, such as indwelling medical devices, that may comprise a core region which may comprise more than 64% of the volume of the indwelling medical device, and a surface region which may comprise less than 36% of the volume of the indwelling medical device.

In certain examples, the surface region may comprise an interpenetrated polymer network which may comprise a primary polymer network and a secondary polymer network, where the interpenetrated polymer network has a thickness of more than 100 nm.

In examples, the primary polymer network may comprise a hydrophobic polymer network. In some examples, the secondary network may comprise a hydrophilic polymer network. In examples, the secondary polymer network may comprise zwitterionic moieties.

In examples, the primary polymer network and the secondary polymer network may not be covalently bonded. In other examples, the primary polymer network and the secondary polymer network may be at least partially covalently bonded.

In examples, the primary polymer network may be selected from the group consisting of segmented polyether polyurethane, thermoplastic polyurethane (TPU), polycarbonate-based TPU, polyether-based TPU, polyester-based TPU, thermoplastic elastomer, thermoplastic olefin, polyester, silicone, latex, polyvinyl chloride, polyethylene terephthalate, polyacrylate, and polymethacrylate.

In examples, the secondary polymer network may be selected from the group consisting of polyurethane, polyacrylate, polymethacrylate, polyvinyl chloride, polyethylene terephthalate, silicone, latex, polyvinyl alcohol, polyethylene glycol, and hyaluronic acid. In some examples, the secondary polymer network may be selected from the group consisting of zwitterionic polymethacrylates, zwitterionic polyacrylates, zwitterionic thermoplastic polyurethane, and zwitterionic silicone.

In examples, the core region may be free of the interpenetrated polymer network.

In examples, the surface region may further comprise a superficial domain and a subsurface interpenetrated network domain, where the superficial domain is the outermost layer of the surface region, where the subsurface interpenetrated network domain lies beneath the superficial domain.

In examples, the superficial domain may be highly hydrophilic and possess a large percentage of zwitterionic moieties, while the subsurface may comprise predominately of the primary polymer network. In some examples, the zwitterionic moieties may be present in both the superficial domain, and the subsurface interpenetrated network domain with a progressively diminishing density of the primary interpenetrated network at increasing depths.

In examples, the surface region may be anti-fouling.

In examples, the surface region may further comprise non-leachable biocides or leachable antibiotics. In some examples, the non-leachable biocides may comprise cationic or gemini dicationic moieties. In examples, the leachable antibiotics may be selected from the group consisting of minocycline, rifampin, chlorohexidine, and combinations thereof. In some examples, the leachable antibiotics may comprise minocycline and rifampin.

In examples, the surface region may be anti-infective. In some examples, the surface region may be antimicrobial. In some examples, the surface region may be anti-fouling, antimicrobial, and anti-infective for more than two weeks. In other examples, the surface region may be anti-fouling, antimicrobial, and anti-infective for more than four weeks.

Disclosed herein are examples of a method of manufacturing an indwelling medical device, that may comprise swelling a medical device in a first solution to facilitate penetration of precursors into the medical device, and submerging the medical device in a second solution to polymerize an interpenetrated polymer network, where the first solution may comprise hydrophobic monomers, radical initiator, and a solvent, the second solution may comprise hydrophilic monomers, catalyst, and a solvent, and the solvent may be configured to form a core region comprising more than 64% of the volume of the indwelling medical device, and a surface region comprising less than 36% of the volume of the indwelling medical device. The surface region may comprise an interpenetrated polymer network comprising a primary polymer network and a secondary polymer network, where the interpenetrated polymer network has a thickness of more than 100 nm.

In examples, the solvents may comprise water-miscible organic solvents or a blend of those solvents.

In examples, the hydrophobic monomers may be crosslinkers and comonomers that may be configured to form a part of the secondary polymer network. In some examples, the hydrophilic monomers may be configured to form the majority of the secondary polymer network.

In examples, the solutions may be configured to form a surface region which may comprise a superficial domain and a subsurface interpenetrated network domain, where the superficial domain is the outermost layer of the surface region, where the subsurface interpenetrated network domain lies beneath the superficial domain.

In examples, the solutions may be configured to form a superficial domain that is highly hydrophilic and possess a large percentage of zwitterionic moieties, and a subsurface that may comprise predominately of the primary polymer network. In some examples, the solutions may be configured to form a surface region where zwitterionic moieties are present in both the superficial domain and the subsurface interpenetrated network domain with a progressively diminishing density of the interpenetrated network at increasing depths.

In examples, the method of manufacturing an indwelling medical device may further comprise submerging the medical device in a solution containing antibiotics or biocides to load or impregnate the medical device with non-leachable biocides or leachable antibiotics. In examples, the non-leachable biocides comprise cationic or gemini dicationic moieties. In some examples, the leachable antibiotics may be selected from the group consisting of minocycline, rifampin, chlorohexidine, and combinations thereof. In some examples, the leachable antibiotics may comprise minocycline and rifampin.

In examples, the method of manufacturing an indwelling medical device may further comprise rinsing and drying the medical device.

### BRIEF DESCRIPTION OF THE DRAWINGS

FIG. 1 depicts a schematic illustration comparing prior art methods to the methods of this application.

FIG. 2 depicts a schematic illustration of the formation of the interpenetrating polymer network between TPU and a zwitterionic polymer.

FIG. 3 depicts XPS depth profiling of a central venous catheter (CVC) surface coated with the zwitterionic IPN of the present application compared to the CVC coated with the Semprus coating.

FIGS. 4A-D depict water droplet contact angle measurement on (A) pristine TPU; (B) TPU with Semprus zwitterionic surface with a thickness less than 100 nm; (C) TPU with zwitterionic IPN surface of the present application with a thickness greater than 100 nm; and (D) TPU with zwitterionic and Gemini dicationic IPN surface of the present application.

FIGS. 5A-C depict SEM images of (A) uncoated CVC; (B) CVC with Semprus zwitterionic surface with a thickness less than 100 nm; and (C) surface with zwitterionic IPN surface of the present application with a thickness greater than 100 nm.

FIGS. 6A-C depict in vitro anti-biofilm efficacy of uncoated and Semprus coated CVC, commercially available antimicrobial Cook Spectrum CVC (rifampin and minocycline hydrochloride), and antimicrobial CVC with the zwitterionic IPN of the present application. Specimens were immersed in PBS at 37° C. for 1 hours, 15 days, and 30 days.

FIGS. 7A-K depicts blood cell adhesion and activation of (A) uncoated CVC; (B) antimicrobial Cook Spectrum CVC (rifampin and minocycline hydrochloride), and (C) antimicrobial CVC with the zwitterionic IPN of the present application.

### DETAILED DESCRIPTION

### Definitions

The following definitions are provided for clarification purposes only and are generally indicative of the described concepts. This list of definitions is not all-inclusive with regard to the concepts necessary to understand the present application. Other definitions may be provided elsewhere in this disclosure. A person skilled in the relevant art will understand that different definitions than those provided in this specification may be employed without substantially changing the essential meaning, overall intent, and broad concepts of the present application.

As used herein, the term “biofilm” refers to the mixtures of biomolecules, biopolymers, exopolysachharides, and other materials that serve to anchor microorganisms to various surfaces. Biofilm may be used to refer to such mixtures both with and without adherent microorganisms.

As used herein, the terms “antibiofouling” and “antifouling” refer to the property of an object, in which it discourages the formation of biofilms and/or the adherence of microorganisms.

As used herein, the term “anti-pathogenic” refers to the property of an object, in which it exhibits cytotoxic, inhibitory, and/or antiproliferative action upon pathogenic microorganisms including viruses, bacteria, yeasts, and molds.

As used herein, the term “gemini” refers to a surfactant with two or more polar head-groups separated by a spacer and two or more hydrophobic tails. For more information on this concept and description of various types of Gemini-Surfactants see Menger, F. M. et al. “Gemini Surfactants” Angew. Chem. Int. Ed. 2000, 39, 1906-1920.

As used herein, the term “zwitterionic” refers to molecules that possess both positive and negative electrical charges within the same molecule. Therefore, zwitterionic compounds are mostly electrically neutral (i.e., the net formal charge is usually zero).

As used herein, the term “noncovalent interpenetrated polymer network” refers to a polymer comprising two or more networks which are at least partially interlaced on a molecular scale but not covalently bonded to each other and cannot be separated unless chemical bonds are broken.

As used herein, the term “semi-interpenetrated polymer network” refers to a polymer comprising two or more networks which are at least partially interlaced on a molecular scale, where the secondary network is partially penetrated and/or partially covalently bonded, and the covalent bonds do not significantly influence the property of the primary network.

As used herein, the terms “interpenetrated polymer network” (“IPN”), “double network,” and “secondary network” refers to both noncovalent interpenetrated polymer network and semi-interpenetrated polymer network. As used herein, the terms “hydrophilic-hydrophobic interpenetrated network” and “hydrophobic-hydrophilic interpenetrated network” are used interchangeably, and refer to interpenetrated networks between hydrophilic and hydrophobic polymers, that are optionally covalently bonded to each other.

As used herein, the terms “coating,” “surface,” and “surface coating” are used interchangeably, and refer to pristine or newly created surfaces and their attributes.

As used herein, the term “silicone” refers to compounds and polymers comprised of chains of alternating silicon atoms and oxygen atoms. Silicone includes, for example, PDMS. As used herein, the terms “DUDMA,” “diurethane-dimethacrylate,” “UDMA,” and “urethane dimethacrylate” may be used interchangeably.

As used herein, the term “PDMS” refers to poly-dimethylsiloxane.

As used herein, the term “PET” refers to polyethylene terephthalate. PET may also be abbreviated as PETE (or the obsolete PETP or PET-P).

As used herein, the term “PVC” refers to polyvinyl chloride.

As used herein, the term “PU” refers to polyurethane.

As used herein, the term “TPU” refers to thermoplastic polyurethane. PU and TPU are used interchangeably. TPU includes, for example, polycarbonate-based TPU, polyether-based TPU, polyester-based TPU, and polyether & polyester-based TPU.

As used herein, the term “PMMA” refers to polymethyl methacrylates.

As used herein, the term “superficial surface” refers to the outermost surface.

As used herein, the term “subsurface” refers to the area below superficial surface.

As used herein, the term “surface” refers to both superficial surface and subsurface.

As used herein, the terms “composition,” “IPN,” “coating,” and “surface,” alone or in combination with any other term(s), may be used interchangeably to refer to the subject of the current application.

The examples set forth herein and recited in the claims can be understood in view of the above definitions.

### Indwelling Medical Devices

Disclosed herein are examples of an indwelling medical device that may comprise a core region which may comprise more than 64% of the volume of the indwelling medical device, and a surface region which may comprise less than 36% of the volume of the indwelling medical device. The surface region may comprise an interpenetrated polymer network which may comprise a primary polymer network and a secondary polymer network, where the interpenetrated polymer network has a thickness of more than 100 nm.

Exemplary indwelling medical devices may achieve high durability by utilizing an interpenetrating polymer network (IPN). In examples, a zwitterionic secondary polymer network and a hydrophobic primary polymer network may be integrated specifically within the surface area of the primary polymer network. The IPN surface may constitute less than 36% of the total thickness of the substrate. In some examples, the IPN surface portion may serve as a coating layer, while the primary polymer may act as the substrate. In examples, the formation of an IPN involves swelling of primary network to facilitate penetration of precursors (monomers and/or comonomers), crosslinkers, and initiators, followed by polymerization of the secondary network.

In examples, the precise portion of IPN (formed between the primary hydrophobic and secondary zwitterionic polymer networks) may dictate the effectiveness of coatings for indwelling medical device applications such as intervascular catheters. When the IPN coating is too thin (less than 100 nm) relative to the total thickness (500 μm) of the primary polymer, the coating may lack durability. Conversely, when the IPN layer is too thick (greater than 100 μm), it may compromise the integrity of the primary network.

In examples, the specific portion and thickness of the IPN may dictate the effectiveness of indwelling medical devices such as intravascular catheters, including peripheral intravenous line (PIV), central venous catheter (CVC), peripherally inserted central catheter (PICC), Jugular Axillo-Subclavian Central Catheter (JACC), and hemodialysis catheter (HDC).

More specifically, in some examples, when the thickness of the IPN layer or portion in a primary thermoplastic polyurethane (TPU, a type of SPU) substrate (used for intervascular catheter) is less than 100 nm, the hydrophilicity of the zwitterionic polymer network lasts for less than 3 days. Therefore, a thin IPN layer/portion or surface (thickness less than 100 nm) is insufficient to maintain long-term hydrophilicity (crucial for biofilm repellency), which is critical for sustained anti-biofilm performance in indwelling medical device applications.

On the other hand, when the thickness of the IPN layer of portion exceeds 100 μm of TPU substrate with 550 μm thickness (used for intervascular catheters), the mechanical properties, such as tensile strength and elongation, of the TPU-based intravascular catheter significantly decrease. The solvent-swelling process necessary to achieve deeper penetration of the secondary network may compromise the integrity of the primary TPU network, leading to a decrease in the catheter's overall mechanical performance (such as tensile strength, hardness, elongation, modulus). Therefore, the thickness of the IPN layer or portion around the surface area relative to the total thickness of substrate should be less than 36%.

In examples, an IPN may have a specific thickness (over 100 nm) and specific surface portion (less than 36% relative to total volume). In examples, the IPN may be a partial or semi-IPN.

In examples, the primary polymer network may comprise a hydrophobic polymer network. In some examples, the secondary network may comprise a hydrophilic polymer network. In examples, the secondary polymer network may comprise zwitterionic moieties. In some examples, the IPN may be formed between a zwitterionic polymer network and a TPU polymer network for indwelling medical device applications such as intravascular catheters.

In examples, the primary polymer network and the secondary polymer network may not be covalently bonded. In other examples, the primary polymer network and the secondary polymer network may be at least partially covalently bonded. In examples, polymeric networks containing zwitterionic moieties may be bound to polymeric substrates such as TPU through covalent bonding, as well as molecular entanglement or interlock or interlace.

In examples, the primary polymer network may be selected from the group consisting of segmented polyether polyurethane, thermoplastic polyurethane (TPU), polycarbonate-based TPU, polyether-based TPU, polyester-based TPU, thermoplastic elastomer, thermoplastic olefin, polyester, silicone, latex, polyvinyl chloride, polyethylene terephthalate, polyacrylate, and polymethacrylate.

In examples, the secondary polymer network may be selected from the group consisting of polyurethane, polyacrylate, polymethacrylate, polyvinyl chloride, polyethylene terephthalate, silicone, latex, polyvinyl alcohol, polyethylene glycol, and hyaluronic acid. In some examples, the secondary polymer network may be selected from the group consisting of zwitterionic polymethacrylates, zwitterionic polyacrylates, zwitterionic thermoplastic polyurethane, and zwitterionic silicone.

In examples, highly hydrophilic zwitterionic polymers may be interpenetrated into hydrophobic polymers such as TPU, silicone, PVC, and latex. In examples, the secondary network containing zwitterionic moieties may be covalently bonded to the primary network. In some examples, antibiotics or biocides may be impregnated into the interpenetrated or interpenetrated-covalently bonded polymeric surfaces.

In examples, the core region may be free of the interpenetrated polymer network.

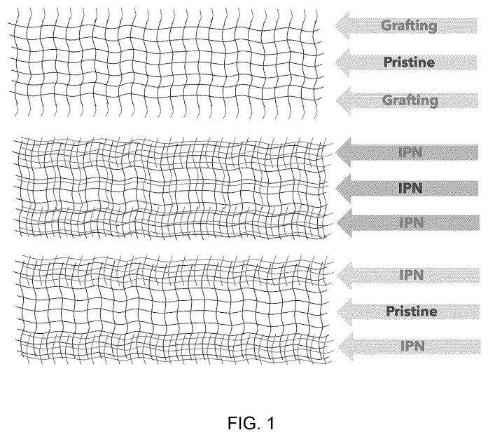

FIG. 1 depicts a schematic illustration comparing prior art methods to the methods of this application. The top schematic illustrates the method of Semprus. Semprus teaches surface grafting using a zwitterionic linear polymer. This method focuses on grafting the zwitterionic polymer onto the surface, providing a surface modification.

The middle schematic illustrates the method of US 2011/0282005A1. US 2011/0282005A1 teaches a functionally graded IPN. The method results in an IPN that has a higher density of the secondary network in the surface area, gradually decreasing through the depth of the material.

The bottom schematic illustrates an example method. The IPN structure is confined to only the surface area, constituting less than 36% of the total volume. The pristine polymer portion is maintained, constituting more than 64% of the total volume. This method achieves a durable zwitterionic surface while maintaining the integrity of the primary polymer network beneath.

In examples, the surface region may further comprise a superficial domain and a subsurface interpenetrated network domain, where the superficial domain is the outermost layer of the surface region, where the subsurface interpenetrated network domain lies beneath the superficial domain.

In examples, the superficial domain may be highly hydrophilic and possess a large percentage of zwitterionic moieties, while the subsurface may comprise predominately of the primary polymer network. In some examples, the zwitterionic moieties may be present in both the superficial domain, and the subsurface interpenetrated network domain with a progressively diminishing density of the primary interpenetrated network at increasing depths.

In some respect, the secondary networks disclosed herein may be thought of as a nanoscale analogue of fiberglass resin composite. In a similar way to how curing of epoxy resin forms a secondary three-dimensional polymer network between the honeycomb of voids in a fiberglass cloth, thereby mechanically/molecularly/topologically locking the polymer/cloth together, secondary networks are formed at the molecular level by the growth of a crosslinked three-dimensional polymer in between the voids between polymer chains of the substrate that is being coated. In some examples, the coatings disclosed herein may differ from simple-double-network polymers by having a higher degree of three-dimensionality providing entanglement with the substrate beneath the surface, while the portion of the coating polymer that extends above the substrate has significantly less three-dimensionality with little to no crosslinking between the superficial polymer chains.

In examples, the superficial- and less-crosslinked portion of the polymer coating may be highly hydrophilic and may possess a large percentage of hydrophilic residues such as zwitterionic residues, while the subsurface and molecularly interlocked secondary network may comprise predominantly of hydrophobic monomers. In some examples, the coatings may comprise zwitterionic moieties that are present in both the superficial domain, and the subsurface interpenetrated network domain with a progressively diminishing density of the interpenetrated network at increasing depths.

In examples, the polymer chains of the coating may form a crosslinked three-dimensional interpenetrated network that is molecularly interlocked/topologically knotted with the substrate below the surface while above the surface the polymer chains of the coating may be uncrosslinked, forming a brush like architecture.

In examples, the surface region may be anti-fouling.

In examples, the surface region may further comprise non-leachable biocides or leachable antibiotics. In some examples, the non-leachable biocides may comprise cationic or gemini dicationic moieties. In examples, the leachable antibiotics may be selected from the group consisting of minocycline, rifampin, chlorohexidine, and combinations thereof. In some examples, the leachable antibiotics may comprise minocycline and rifampin.

In examples, the secondary network may contain both zwitterionic moieties and cationic (or gemini dicationic) moieties to achieve antimicrobial efficacy in addition to antifouling activity.

In examples, drugs such as antibiotics or biocides may be impregnated into the IPN surface of the indwelling medical device. It has been found that the three-dimensional structure of the IPN surface provides an extended drug release profile of antibiotics, such as minocycline and rifampin. In some examples, the IPN may comprise antibiotics selected from the group consisting of minocycline, rifampin, chlorohexidine, and their combinations.

In examples, the hydrophilic-hydrophobic IPN may be used to create a surface or surface coating with antimicrobial, anti-biofilm, anti-thrombosis, or anti-infection properties. In examples, the surface region may be anti-infective. In some examples, the surface region may be antimicrobial. In some examples, the surface region may be anti-fouling, antimicrobial, and anti-infective for more than two weeks. In other examples, the surface region may be anti-fouling, antimicrobial, and anti-infective for more than four weeks. In examples, the polymer composition may ensure sustained and durable anti-fouling and antimicrobial activities for over 30 days, resulting in a reduction in infections associated with indwelling medical devices, such as catheter-related infections.

In examples, the IPN portion may be formed between a TPU network and a zwitterionic polymer network. This composition maintains the mechanical integrity of the TPU substrate while providing excellent anti-fouling surface activity. This composition demonstrated extended anti-fouling, antimicrobial, and anti-infective properties, outperforming state-of-the-art zwitterionic surfaces.

In examples, these surfaces are used for antifouling, anti-biofilm, anti-infective, antimicrobial, or fouling-release surfaces, coatings, medical devices, or catheters. In some examples, the surfaces or coatings disclosed herein prevent biofouling or biofilm formation on medical devices such as catheters. In another example, the surfaces generated on medical devices reduce the incidence of nosocomial infections. In examples, the surfaces may comprise TPU, silicone, PVC or latex. In some examples, catheters with such surfaces may reside in biological systems such as in human body with lower risk of associated infections.

Disclosed herein are examples of a method of manufacturing an indwelling medical device, that may comprise swelling a medical device in a first solution to facilitate penetration of precursors into the medical device, and submerging the medical device in a second solution to polymerize an interpenetrated polymer network, where the first solution may comprise hydrophobic monomers, radical initiator, and a solvent, the second solution may comprise hydrophilic monomers, catalyst, and a solvent, and the solvent may be configured to form a core region comprising more than 64% of the volume of the indwelling medical device, and a surface region comprising less than 36% of the volume of the indwelling medical device. The surface region may comprise an interpenetrated polymer network comprising a primary polymer network and a secondary polymer network, where the interpenetrated polymer network has a thickness of more than 100 nm.

In examples, the solvents may comprise water-miscible organic solvents or a blend of those solvents. In examples, the IPN may be prepared using water-miscible organic solvents or a blend of those solvents. In some examples, the process of preparing the IPN may comprise swelling a hydrophobic network to facilitate the incorporation of crosslinkers, precursors, initiators, or catalysts. Subsequently, those components (i.e., crosslinkers, precursors, initiators, or catalysts) may undergo a reaction to establish a secondary hydrophilic network within a primary hydrophobic network.

In examples, the hydrophobic monomers may be crosslinkers and comonomers that may be configured to form a part of the secondary polymer network. In some examples, the hydrophilic monomers may be configured to form the majority of the secondary polymer network.

In examples, the solutions may be configured to form a surface region which may comprise a superficial domain and a subsurface interpenetrated network domain, where the superficial domain is the outermost layer of the surface region, where the subsurface interpenetrated network domain lies beneath the superficial domain.

In examples, the solutions may be configured to form a superficial domain that is highly hydrophilic and possess a large percentage of zwitterionic moieties, and a subsurface that may comprise predominately of the primary polymer network. In some examples, the solutions may be configured to form a surface region where zwitterionic moieties are present in both the superficial domain and the subsurface interpenetrated network domain with a progressively diminishing density of the interpenetrated network at increasing depths.

In examples, the method of manufacturing an indwelling medical device may further comprise submerging the medical device in a solution containing antibiotics or biocides to load or impregnate the medical device with non-leachable biocides or leachable antibiotics. In examples, the non-leachable biocides comprise cationic or gemini dicationic moieties. In some examples, the leachable antibiotics may be selected from the group consisting of minocycline, rifampin, chlorohexidine, and combinations thereof. In some examples, the leachable antibiotics may comprise minocycline and rifampin.

In examples, the method of manufacturing an indwelling medical device may further comprise rinsing and drying the medical device.

### EXAMPLES

Some examples discussed above are disclosed in further detail in the following examples, which are not in any way intended to limit the scope of the present disclosure. Those in the art will appreciate that many other examples also fall within the scope of the compositions and related methods of the present application, as described herein above and in the claims.

The following examples describe preparation of some examples of the present application. Commercially available starting materials and reagents were used in preparing these compositions, such as those from the Aldrich Chemical Company (Milwaukee, Wis.), Bachem (Torrance, Calif.), Sigma (St. Louis, Mo.), or were prepared by methods well known to a person of ordinary skill in the art.

### Example 1: Exemplary Procedures for Forming Molecularly Interlocked Antifouling Surfaces or Surface Coatings on Biomedical Polyurethane

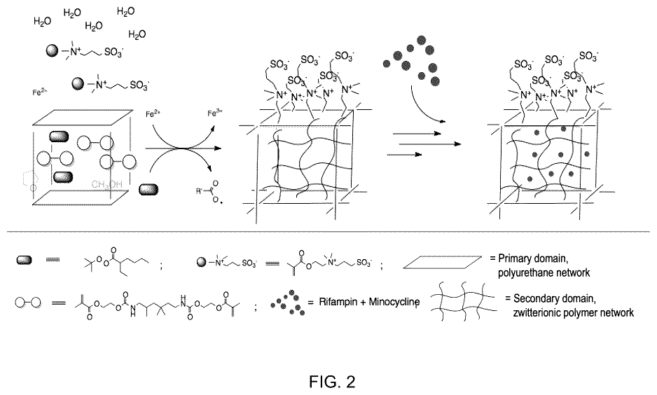

FIG. 2 depicts a schematic illustration of the formation of the interpenetrating polymer network between TPU and a zwitterionic polymer. FIG. 2 illustrates how 1) surfaces or surface coatings of the present application are formed, 2) surfaces or surface coatings of the present application are molecularly interlocked, 3) some the chemical processes described influence the properties of the resulting surfaces or surface coatings of the present application 3) surface or surface coatings of the present application have differing functionalities and differing polymer architecture depending upon location within the substrate.

Specific monomers, surface or surface coating topology, surface or surface coating stratification used here are for clarity and are not meant to restrict or depart from the broad concepts. For instance, one skilled in the art will recognize that different monomers, catalysts, and solvents, and reaction conditions may be employed to change the properties of the resulting surface or surface coating. Some aspects of the substrates structure are removed for clarity to assist in visualization, for instance, the polyurethane substrate is depicted as two vertically stacked planes while actual biomedical polyurethanes are three-dimensional networks.

In examples, the choice of swelling solvent used to load the hydrophobic monomers influences the depth at which hydrophilic functional monomers may be incorporated into the surface or surface coating. For instance, loading hydrophobic monomers by swelling in water miscible organic solvents/mixtures enables diffusion/back-diffusion of both hydrophobic and hydrophilic monomers during the surface or surface coating process allowing the functional monomers to be incorporated into the surface or surface coating at greater subsurface depths. It is noted that the properties of the resulting surface or surface coatings may change as a result of the degree of evaporation of solvents/hydrophobic monomers once a substrate has been removed from a swelling solution.

In examples, the present application teaches surface or surface coatings with an interphase region of both functional-hydrophilic and anchoring-hydrophobic monomers. In some examples, the interphase region permits for a higher density of functional monomers to be incorporated into the surface or surface coating. In other examples, the interphase region during polymerization allows polymerization to proceed with a gradual transition from hydrophobic to hydrophilic domains without incurring sudden unfavorable entropic penalties.

### Sample Preparation and Cleaning

Uncoated, polyurethane catheters were cut with a razor blade into 1 cm long segments. Approximately 50 catheter segments were placed into a 50 mL polypropylene centrifuge tube. Samples were rinsed sequentially with 3×40 mL deionized water then 3×40 mL 200 proof EtOH as follows: 40 mL of deionized water was added to the centrifuge tube, the tube was then capped and gently agitated until all the catheter segments no longer contained trapped air bubbles causing them to float. Once all the segments settled to the bottom of the tube, the tube was shaken gently for an additional 3 minutes, then allowed to stand until all the segments had settled to the bottom of the tube. The water was decanted off, leaving no more than 4 mL of residual liquid. The process was repeated 2 more times, then the same process was repeated 3 more times with 200 proof EtOH. The catheter segments were removed from the centrifuge tube with a PTFE coated spatula and spread evenly on a sheet of filter paper to dry at ambient temperature.

### Preparation of Interpenetrating Monomer Solution A

“Solution A”, a solution of monomers for loading of subsurface hydrophobic monomers, was prepared first as follows: To a clean, dry, 250 mL glass culture flask was added 3 g of diurethane-dimethacrylate (e.g. DUDMA, Sigma Aldrich, CAS #72869-86-4) followed by 80 mL of MeOH and 20 mL of THF. A clean, 1 inch PTFE coated stir bar vial was then added, and the flask was capped tightly and stirred magnetically until all the DUDMA had fully dissolved. Subsequently, the cap was removed and 0.15 mL of trimethylolpropane trimethacrylate (e.g. TMPTMA, CAS #3290-92-4, Supplier: TCI) and 0.3 mL of 2-Ethylhexyl methacrylate CAS #688-84-6) was added via pipette. The flask was then resealed, and the solution was stirred vigorously for 2 minutes to give approximately 103 mL of Solution A.

### Preparation of Interpenetrating Monomer Solution B

“Solution B”, was prepared from Solution A by addition of a peroxide polymerization initiator as follows: To the preceding flask containing 103 mL of freshly prepared Solution A was added 3 mL of tert-butylperoxy 2-ethylhexyl carbonate (e.g. TBEC, CAS #34443-12-4, Sigma Aldrich) via syringe. The flask was sealed, wrapped tightly in aluminum foil to protect the solution from light, and the solution was stirred for 3 minutes at ambient temperature.

Loading of Subsurface Monomers (Swelling with Hydrophobic Monomers and Radical Initiator)

Once no further odor of EtOH was detectable in Solution B, the freshly cut, cleaned and air-dried catheter segments (approx. 50×1 cm-long samples) were transferred from the filter paper to a fresh 50 mL polypropylene centrifuge tube, and 30 mL of freshly prepared Solution B was added. The tube was capped tightly and shaken vigorously until all samples were observed to settle to the bottom of the vial when shaking was ceased, indicating that no air bubbles were trapped in the tube that would prevent contact with the solution. The tube was then wrapped in aluminum foil to protect the contents from ambient light and left to sit at ambient temperature for 2 hours, swirling/agitating the tube briefly every 20 minutes.

### Preparation of Zwitterionic Monomer Solution C

To a fresh, tared, 50 mL polypropylene centrifuge tube was added a small (approx. ¼″), conical, PTFE coated magnetic spin vane, followed by 4 g of 3-[[2-(Methacryloyloxy)ethyl]dimethylammonio]propane-1-sulfonate (CAS #3637-26-1, obtained from TCI) and 40 mL of DI water. The vial was capped tightly, shaken vigorously until all of the monomer had dissolved, then set aside.

### Preparation of Zwitterionic and Cationic Monomers Solution D

“Solution D” was prepared by exchanging the iodide counterions of a diquaternary ammonium methacrylate monomer (“Gemini” diquaternary-ammonium methacrylate) for chloride anions, then dissolving zwitterionic methacrylate in the freshly prepared solution of diquaternary ammonium dichloride methacrylate as follows: 52.4 mg of diquaternary ammonium iodide methacrylate MA-2-3-18·2I− was added to a 20 mL amber glass vial, followed by 1 g of Dowex 1×8 ion exchange resin (chloride form), and 10 mL of DI water. The vial was capped, then vortexed vigorously for 15 minutes during which the solution became transparent, indicating the formation of the dichloride MA-2-3-18·2Cl—. The resin was filtered off through a glass fritted funnel into a tared 50 mL polypropylene centrifuge tube. The resin was rinsed with 3×8.666 mL DI water then additional DI water as necessary to bring the total volume of filtrate to 40 mL. To the centrifuge tube containing the filtrate was then added a small (approx. ¼″), conical, PTFE coated magnetic spin vane and 4 g of 3-[[2-(Methacryloyloxy)ethyl]dimethylammonio]propane-1-sulfonate. The vial was capped and shaken vigorously until all solids had dissolved, then set aside.

### Preparation of PU-Z and PU-Z+Gemini Samples (Surface Grafting of Hydrophilic Monomers)

The catheter segments swollen with the solvent/monomers/initiators of Solution B were then immediately subjected to surface modification.

A water bath was prepared by insulating a 500 mL borosilicate glass beaker with a strip of cotton/polypropylene fabric, which was secured with several wrappings of PTFE Tape. The beaker was then wrapped with 2 layers of aluminum foil which was secured with black vinyl electrical tape. Approx. 200 ml of water and a small ¾″ oval magnetic stir bar were added to the beaker, and the beaker was placed on top of a dual magnetic stirring/hotplate with a temperature probe/thermocouple. The temperature probe was then placed 1″ below the water level in the center of the beaker. Stirring and heating of the water bath was then commenced setting the stirring at a rate of 120 rpm and temperature of the bath to 60° C. Excessive stirring was avoided to minimize potential abrasion.

Once the water bath was prepared/heating, 384 mg of iron (II) gluconate was weighed into a tared vial, capped, and the vial was set aside.

Once the catheter segments had been soaked for 2 hours in Solution B. The catheter segments were filtered off quickly (<2 minutes) by pouring the contents of the centrifuge tube through a coarse fritted funnel. The segments were then transferred to a folded piece of filter paper, blotted gently with a Kimwipe to remove droplets of Solution B still clinging to the catheter segments, then transferred to a 50 mL centrifuge tube containing 40 mL of freshly prepared Solution C or Solution D. The total time elapsed for filtering excess Solution B, blotting, and transferring swollen samples to Solution C or D did not exceed 5 minutes.

384 mg of pre-weighed iron (II) gluconate was then added to the submerged catheter samples in Solution C or D, the tube was capped tightly and shaken vigorously by hand for several minutes until all of the iron (II) gluconate had dissolved. The sealed vial containing Solution C or D, catheter segments, and iron (II) gluconate was then placed into the preheated water bath and left to stir gently at 60° C. for 4 hours.

The surface coating process could also be conducted without disadvantage by grafting at 50° C. for 8 hours, or 40° C. for 16 hours. The relative volume of air (the headspace) to the volume of the grafting solution in the sealed vial was an important parameter for successful grafting. Running the procedure in sealed tubes with <5% headspace led to competitive solution phase polymerization of the soluble monomers that diminished the quantity advisable to undergo surface-grafting polymerization, while running the procedure in vials open to air or sealed vials with >50% headspace relative to volume of the solution led to insufficient degree of grafting polymerization. This is consistent with the well-known inhibitory effect of molecular oxygen on radical polymerizations. As such, it is important to maintain a tight seal, and maintain a headspace of approximately 20% of the vessels volume to achieve reproducible surface grafting under these conditions.

After 4 hours had elapsed, the centrifuge tube was removed from the water bath, and the liquid was decanted off, leaving the catheter segments in the tube. To remove unbound monomers and ungrafted polymers from the samples, the catheter segments were rinsed sequentially with 8×40 mL of 0.9% wt/v NaCl in DI water, then 3×40 ml DI water, then 3×40 ml MeOH, by adding the rinsing solution to the vial, shaking vigorously for 30 seconds, then decanting off the liquid. After the last of the liquid had been decanted off, 40 mL of 0.9% wt/v NaCl was added and the vial was left to stand for 4 days at ambient temperature, decanting off the NaCl solution and replenishing with fresh solution every 24 hours. Traces of NaCl were removed by shaking/decanting with 40 mL of DI water, and the samples were then soaked in 40 mL of MeOH for 48 hours to remove any unreacted monomers still swollen within the samples. The samples were collected by suction filtration on a Buchner funnel, transferred to a clean piece of filter paper, and the coated samples were left to air dry for 48 hours.

Coating polyurethane catheter segments with Solution C resulted in zwitterionic coated polyurethane samples (PU-Z). Coating polyurethane catheter segments with Solution D resulted in zwitterionic and gemini dicationic coated polyurethane samples (PU-Z+Gemini).

Preparation of PU-Z+R/M Samples (Zwitterionic-Coated Polyurethane with Leachable Rifampin/Minocycline)

PU-Z catheter segments were loaded with antibiotics as follows: 50 PU-Z catheter segments were added to a 50 mL centrifuge tube containing 25 mL of a freshly prepared solution of rifampin (2 mg/mL) and minocycline hydrochloride (2 mg/mL) in 80:20 v/v MeOH/THF. The vial was sealed and shaken vigorously to remove entrapped air bubbles within the tubes. Once all segments were observed to settle, the vial was wrapped in aluminum foil to protect from light, and the samples were left to swell/stand for 2 hours with occasional swirling. After 2 hours, the catheter segments were filtered to dryness on a suction frit, gently moving the segments around with a PTFE coated spatula to prevent them from clinging to one another and to facilitate the removal of liquid held inside the samples by capillary action. Samples were then rinsed rapidly three times with 20 mL of 200 proof EtOH, dried on the suction-frit, then transferred to a clean piece of filter paper and left to air dry in the dark for 48 hours, resulting in samples of PU-Z+R/M. Samples of PU-Z+R/M were easily distinguishable from PU-Z samples due to the orange color of rifampin/minocycline, qualitatively confirming successful loading of the antimicrobials.

Illustrative Synthesis of a Gemini Dicationic Methacrylate (MA-2-3-18·2I⁻) N¹-(2-(methacryloyloxy)ethyl)-N¹,N¹,N³,N³-tetramethyl-N³-octadecylpropane-1,3-diaminium.

To a dry 250 mL round bottom flask equipped with a rubber septa, PTFE coated magnetic stir bar, and rubber septa, under an atmosphere of dry Argon, was added 150 mL of anhydrous acetone, and 40 mmol (2 equiv.) of 1,3-diiodopropane (CAS #124-28-7, Tokyo Chemical Industry Co.), that was colorless and free of HI and molecular iodine impurities. The flask was cooled with stirring to 0° C. for 10 minutes, whereupon 20 mmol of N,N-dimethyl-octadecyl-1-amine (CAS #124-28-7, Tokyo Chemical Industry Co.) was added slowly via syringe. The septa was tightly sealed with several layers each of PTFE tape, electrical tape, and parafilm, then the flask was removed from the bath and protected from light by wrapping the flask tightly with aluminum foil. The mixture was then left to stirred in the dark at ambient temperature for 72 hours. After 72 hours had elapsed, the aluminum foil was removed revealing production of a voluminous white precipitate of 1-(3-iodopropyl)-octadecyl dimethylammonium iodide. The septa was removed and the contents of the flask were diluted with anhydrous acetone and the product was collected by vacuum filtration. The crude product was rinsed with copious amounts of anhydrous acetone until TLC analysis showed that no residual diiodopropane or amine remained. Entrapped volatiles were removed under reduced pressure and the material was stored protected from light/moisture in the refrigerator in tightly sealed vials until further use.

To a dry 250 mL round bottom flask equipped with a rubber septa, PTFE coated magnetic stir bar, and rubber septa, under an atmosphere of dry air, was added 5 mmol of the 1-(3-iodopropyl)-octadecyl dimethylammonium iodide, 30 mL of anhydrous acetonitrile, and 15 mmol of 2-(dimethylamino)ethyl methacrylate (CAS #2867-47-2, Sigma Aldrich), and 50 mg of butylated hydroxytoluene as radical inhibitor. The flask was sealed then placed into a heating bath atop a magnetic stir plate and heated to 50° C. with stirring. Additional anhydrous MeCN was added slowly in 1 mL increments to the hot solution until all of the 1-(3-iodopropyl)-octadecyl dimethylammonium iodide had dissolved. The vessel was then protected from light with aluminum foil and left to stir for 18 hours at 50° C. The aluminum foil was removed revealing production of a white precipitate of MA-2-3-18·2I⁻, and the solution was filtered while hot and the crude material was rinsed with acetone until no further odor of the amine could be detected. The crude material was further purified by fractional recrystallization with MeCN/acetone. The combined filtrates were concentrated by rotary evaporation and triturated with acetone to remove excess amine/BHT. Traces of unreacted 1-(3-iodopropyl)-octadecyl dimethylammonium iodide were removed by fractional recrystallization with MeCN/acetone. The combined yield of MA-2-3-18·2I⁻ was approximately 45% as a white powder that was only sparingly soluble in water.

### Example 2: Alternative Exemplary Procedures for Forming Molecularly Interlocked Antifouling Surfaces or Surface Coatings on Biomedical Polyurethane

Example 1 is only one example of many methods to prepare the IPN surfaces disclosed herein. In another aspect, the TPU catheter with 750 μm thickness was swelled in a swelling/incubation solution for hours, optionally at <0° C. The swelling/incubation solution may contain any zwitterionic monomer such as sulfobetaine methacrylate (SBMA), any alkyl methacrylate such as 2-ethylhexyl methacrylate (EHMA) monomer to give flexibility to the backbone, any bismethacrylate crosslinker such as DUDMA or glycerol 1,3-diglycerolate diacrylate, and any free radical polymerization initiator, such as azobisisobutyronitrile (AIBN).

In another aspect, the solution may contain, any Reversible Addition Fragmentation Chain Transfer (RAFT) agent for RAFT polymerization, such as trithiocarbonates. In another aspect, the solution may contain any Atom Transfer Radical Polymerization (ATRP) initiator and ATRP catalyst. ATRP initiators are often bromide-containing alkyl halides, such as 2-bromoisobutyryl bromide to form the ATRP reactive sites. Common catalysts include transition metal complexes, such as copper-based complexes, with Cu(I) as the active species. Additionally, the transition metal complexes may employ ligands such as nitrogen containing ligands like 2,2′-bipyridine that coordinate with the metal catalyst.

### Preparation of PU-Z Samples (ATRP Mediated Surface Grafting of Hydrophilic Monomers)

Catheter segments were swelled with SBMA as described in Example 1. A reaction mixture was formed with the ratio of Cu(I) Br (0.1 mmol) and 2,2′-bipyridine (0.2 mmol) per SBMA (10 mmol) in a mixed solvent. The reaction mixture was degassed to remove oxygen. Solvent polarity was varied by using a mixed solvent containing water, methanol, ethanol, isopropanol and/or THF. The polymerization was then conducted for 24 hours.

Once polymerization was complete, the samples were rinsed with DI water and EtOH, and residual monomers were extracted out with 0.9% NaCl overnight and the remaining product was dried. This process was repeated to remove any unreacted residues.

Preparation of PU-Z+R/M Samples (Zwitterionic-Coated Polyurethane with Leachable Rifampin/Minocycline)

Antimicrobials were impregnated by the following process: The catheter segments (5 mm) were impregnated with a mixture of minocycline (15 mg/mL) and rifampin (30 mg/mL) for 1 hour. Optionally, catheter segments (5 mm) were impregnated in a chlorhexidine solution (40 mg/mL) for 4 hours prior to impregnation of minocycline and rifampin (1:2 ratio). After impregnation, catheters are air flushed, dried at 55° C. overnight, washed with water, and dried again. The catheters impregnated with minocycline and rifampin had average concentrations of 503 μg/cm minocycline and 480 μg/cm rifampin for a 7 Fr catheter.

### Example 3: Surface Properties of Zwitterionic IPN

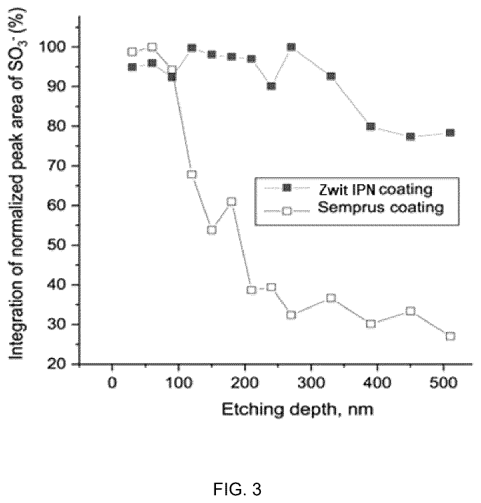

FIG. 3 depicts XPS depth profiling of a central venous catheter (CVC) surface coated with the zwitterionic IPN of the present application compared to the CVC coated with the Semprus coating. The XPS depth profiling results revealed that >80% of zwitterionic moieties in the present zwitterionic-interpenetrated CVC are concentrated within a depth of 500 nm. In contrast, zwitterionic moieties on the Semprus zwitterionic CVC exhibited a significant drop under 100 nm depth and account for <40% under 200 nm.

Additionally, XPS peaks appear at 166.4 eV (2p_{1/2}) and 167.8 eV (2p_{3/2}), characteristic of the sulfonate of the zwitterionic network of the present application. The XPS study shows that in the interpenetrated zwitterionic surface or surface coating of the present application, zwitterionic moieties (S═O) exist at the sub-surface region over 100 nm below the surface (the thickness of interpenetrated network surface or surface coating is greater than 100 nm). In contrast, zwitterionic surface prepared with Semprus methods showed that the zwitterionic moieties (S═O) exist only at the superficial surface less than 100 nm from the surface (the thickness of Semprus surface coating is less 100 nm).

### Example 4: Water Droplet Contact Angle Measurement of Zwitterionic IPN

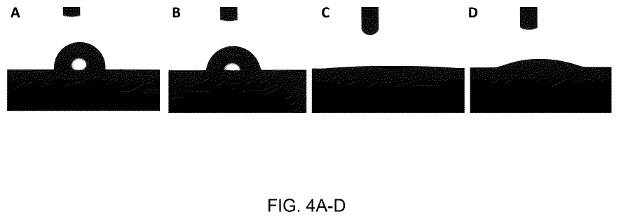

FIG. 4A-D depicts water droplet contact angle measurement on (FIG. 4A) pristine TPU; (FIG. 4B) TPU with Semprus zwitterionic surface with a thickness less than 100 nm; (FIG. 4C) TPU with zwitterionic IPN surface of the present application with a thickness greater than 100 nm; and (FIG. 4D) TPU with zwitterionic and Gemini dicationic IPN surface of the present application. After soaking the surfaces in water for 3 days, water droplet contact angle was measured. The Semprus surface (FIG. 4B) shows ˜86° of water contact angle, suggesting low molecular density of zwitterionic moieties on the surface as its water droplet contact angle (˜86°) is only slightly different from pristine TPU surface (FIG. 4A) with ˜95° of water contact angle. In contrast, zwitterionic IPN of the present application (FIG. 4C) presents ˜0° of water contact angle. This demonstrated that the Semprus surface (FIG. 4B) lost its surface hydrophilicity, due to age-related hydrophobic recovery, after 3 days of soaking in water, whereas zwitterionic IPN of the present application (FIG. 4C) showed stable and durable superhydrophilic surface properties. Further, zwitterionic and Gemini dicationic IPN surface (D) showed ˜3° of water contact angle.

### Example 5: Depth Profiling of Zwitterionic IPN

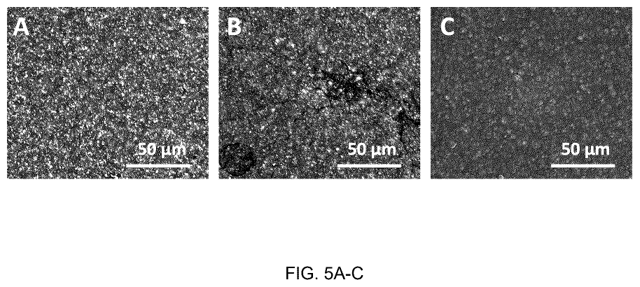

FIG. 5A-C depicts SEM images of (FIG. 5A) uncoated CVC; (FIG. 5B) CVC with Semprus zwitterionic surface with a thickness less than 100 nm; and (FIG. 5C) surface with zwitterionic IPN surface of the present application with a thickness greater than 100 nm. SEM of the CVC surface with Semprus coating (FIG. 5B) revealed a morphology similar to that of the pristine CVC surface (FIG. 5A). In contrast, the SEM image of the CVC surface with zwitterionic IPN coating of the present application clearly displays a significantly different surface morphology compared to both the uncoated CVC (FIG. 5A) and the Semprus coated CVC (FIG. 5B). This distinction suggests a notably higher density of zwitterionic chains on the surface, surpassing the other examples.

In Energy-Dispersive X-ray Spectroscopy (EDS) analysis, silicon-labeled bismethacrylate crosslinkers were utilized to confirm the presence of a secondary network (polymethacrylate) intertwined with the primary network (polyurethane) near and below the surface of the CVC. EDS detected a silicon signal at a depth of 10-60 μm, revealing that the secondary network intertwines with the primary network at the subsurface area.

### Example 6: Biofilm Viability (Anti-Biofilm Efficacy) and Proliferation of Zwitterionic IPN

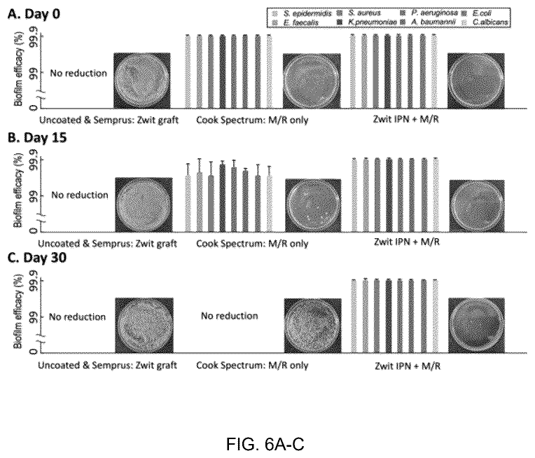

FIG. 6A-C depicts in vitro anti-biofilm efficacy of uncoated and Semprus coated CVC, commercially available antimicrobial Cook Spectrum CVC (rifampin and minocycline hydrochloride), and antimicrobial CVC with the zwitterionic IPN of the present application. Specimens were immersed in PBS at 37° C. for (FIG. 6A) 1 hours, (FIG. 6B) 15 days, and (FIG. 6C) 30 days. At each time point, catheters were immersed in human blood plasma, subsequently inoculated with pathogens, then incubated at 37° C. for 24 hours to allow mature biofilm formation. Loose pathogens were rinsed away, and the segments were reinserted to a plate with fresh media, to seed bacteria into each well. The segments were then removed, additional media was added, and the samples were incubated. While the Cook Spectrum antimicrobial CVC showed ≥99% antibiofilm efficacy over 15 days, the antimicrobial CVC with the zwitterionic IPN of the present application presented ≥99.9% anti-biofilm efficacy over 30 days (≥3 log reduction, which is generally considered significant antimicrobial activity by the FDA).

### Example 7: Antimicrobial Durability

The biofilm viability assay described above was conducted after abrasion challenges. The antimicrobial CVC with the zwitterionic IPN of the present application maintained ≥99.9% of surface antibiofilm efficacy.

### Example 8: In Vitro Cell Viability Assays

In vitro cell viability assays were conducted in accordance with ISO 10993-5 and 10993-17 standards. Briefly, CVC segments were extracted using Dulbecco's Modified Eagle Medium with fetal calf serum for 24, 48, and 72-hour intervals at 37° C. HaCaT keratinocytes were incubated for 48 hours in the presence of the extract, and cell viability was assessed using the Cell Count Kit-8 (Sigma Aldrich). All CVCs exhibited no dead cells, affirming the biocompatibility of the antimicrobial CVC with the zwitterionic IPN of the present application.

### Example 9: Hemocompatibility Tests for Complement Activation, Hemolysis, Coagulation, and Inflammation

Hemocompatibility tests were conducted using the assays described above for complement activation, hemolysis, coagulation, and inflammation in accordance with guidelines set forth in ISO 10993-4 using standard kits according to the manufacturers' instructions. Hemolysis was determined with the QuantiChrome Hemoglobin assay kit (Bioassay Systems, Hayward, CA). According to ASTM F756-17, a hemolytic index of >5% is classified as “hemolytic.” All CVCs showed “non-hemolytic” properties, with median hemolytic indexes ranging from 0.6 to 0.9%. Complement activation of C3a and sC5b-9 was determined using the C3a kit and sC5b-9 Plus EIA kit, (Microvue, San Diego, CA). C3a and sC5b-9 levels showed no statistical differences between all CVCs. To assess the acute inflammatory reaction caused by leukocyte and thrombocyte activation or membrane disruption, we analyzed IL-8, TNF-α, and VEGF released from leukocytes into plasma upon exposure to CVCs using V-PLEX Plus Proinflammatory Panel 1 and V-PLEX Plus Human VEGF kits (MSD, Rockville MD). All CVCs showed no statistical difference.

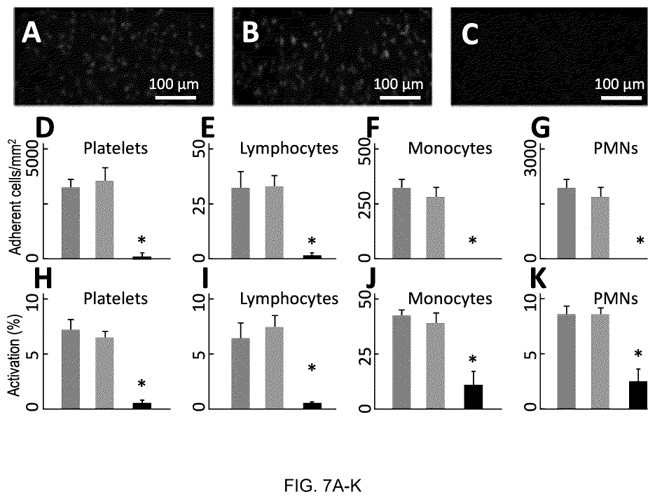

To further demonstrate hemocompatibility, cellular activation and adhesion to CVC surfaces was evaluated, as they play pivotal roles in thrombus formation and inflammatory responses. FIG. 7A-K depicts blood cell adhesion and activation of) uncoated CVC, antimicrobial Cook Spectrum CVC (rifampin and minocycline hydrochloride), and antimicrobial CVC with the zwitterionic IPN of the present application.

A blood perfusion system was employed to assess the adhesion capacity of human platelets, lymphocytes, monocytes, and polymorphonuclear leukocytes (PMNs) to the surface of the CVCs. In this experiment, human blood was flowed over CVC samples for 5 minutes at 37° C., followed by immunostaining of adherent cells (FIG. 7A-C), and quantified using confocal images (FIG. 7D-G). FIG. 7D-G each show uncoated CVC on the right, Cook Spectrum CVC in the middle, and antimicrobial CVC with the zwitterionic IPN of the present application on the right. Antimicrobial CVC with the zwitterionic IPN of the present application exhibited a >90% reduction in cell attachment across all four cell types compared to uncoated CVC and Cook Spectrum CVC. Blood cell activation was evaluated, according to the previously described method (FIG. 7H-K). FIG. 7H-K each show uncoated CVC on the right, Cook Spectrum CVC in the middle, and antimicrobial CVC with the zwitterionic IPN of the present application on the right. The results demonstrated that antimicrobial CVC with the zwitterionic IPN of the present application induced significantly less cell activation compared to uncoated CVC and Cook Spectrum CVC.

### Example 10: Rat Studies for Biocompatibility and Infection

Two groups of 20 rats per group were studied: Group 1 received the CVC with the zwitterionic IPN of the present application and Group 2 received the commercially available Cook Spectrum antimicrobial CVC. Regarding non-superior biocompatibility (safety), weight loss/gain was monitored. The initial difference in body weight between the groups did not show any statistical significance (P>0.7). However, the average daily increase in body weight during the vascular access period in Group 1 was notably higher than that in Group 2 (P<0.01). After a 45-day patency, a significant difference persisted (P<0.001). During follow up, Group 2 had 16 infectious complications occur, whereas Group 1 had 3 infectious complications occur. The infectious complication rate in Group 1 was significantly lower than that of Group 2 (P<0.001). Infections were evaluated by culturing microbes from the skin at exit-site and tip of tube, then the strains were identified using API® ID strip (Table 1). Blood samples were drawn, centrifuged, and plasma was separated before running HPLC. HPLC confirmed that there were no new small molecules (leachates) other than rifampin and minocycline.

TABLE 1
Causative organisms of exit site infection
and catheter tip colonization

| | Group 1: CVC with | |
|---|---|---|
| Location | zwitterionic IPN of current | Group 2: Cook |
| Pathogens identified | application | Spectrum CVC |
| Exit sites | | |
| S. aureus | 1 | 4 |
| S. epidermidis | 1 | 3 |
| Streptococcus spp. | 0 | 1 |
| Escherichia coli | 0 | 1 |
| P. aeruginosa | 0 | 1 |
| Catheter tips | | |
| S. epidermidis | 0 | 4 |
| S. aureus | 1 | 3 |
| Candida | 0 | 2 |
| P. aeruginosa | 0 | 1 |
| E. faecalis | 0 | 1 |

Although the present disclosure includes certain examples and applications, it will be understood by those skilled in the art that the present disclosure extends beyond the specifically disclosed examples to other alternative examples and/or uses and obvious modifications and equivalents thereof, including examples which do not provide all of the features and advantages set forth herein. Accordingly, the scope of the present disclosure is not intended to be limited by the specific disclosures of examples herein, and may be defined by claims as presented herein or as presented in the future.

What is claimed is:

1. An indwelling medical device, comprising:
a core region comprising more than 64% of the volume of the indwelling medical device; and
a surface region comprising less than 36% of the volume of the indwelling medical device, the surface region comprising an interpenetrated polymer network comprising a primary polymer network and a secondary polymer network, wherein the interpenetrated polymer network has a thickness of more than 100 nm.

2. The medical device of claim 1, wherein the primary polymer network comprises a hydrophobic polymer network.

3. The medical device of claim 1, wherein the secondary polymer network comprises a hydrophilic polymer network.

4. The medical device of claim 1, wherein the secondary polymer network comprises zwitterionic moieties.

5. The medical device of claim 1, wherein the primary polymer network is selected from the group consisting of segmented polyether polyurethane, thermoplastic polyurethane (TPU), polycarbonate-based TPU, polyether-based TPU, polyester-based TPU, thermoplastic elastomer, thermoplastic olefin, polyester, silicone, latex, polyvinyl chloride, polyethylene terephthalate, polyacrylate, and polymethacrylate.

6. The medical device of claim 1, wherein the secondary polymer network is selected from the group consisting of polyurethane, polyacrylate, polymethacrylate, polyvinyl chloride, polyethylene terephthalate, silicone, latex, polyvinyl alcohol, polyethylene glycol, and hyaluronic acid.

7. The medical device of claim 1, wherein the secondary polymer network is selected from the group consisting of zwitterionic polymethacrylates, zwitterionic polyacrylates, zwitterionic thermoplastic polyurethane, and zwitterionic silicone.

8. The medical device of claim 1, wherein the core region is free of the interpenetrated polymer network.

9. The medical device of claim 1, wherein surface region further comprises a superficial domain and a subsurface interpenetrated network domain, wherein the superficial domain is the outermost layer of the surface region, wherein the subsurface interpenetrated network domain lies beneath the superficial domain.

10. The medical device of claim 1, wherein the superficial domain is highly hydrophilic and possess a large percentage of zwitterionic moieties, while the subsurface comprises predominately of the primary polymer network.

11. The medical device of claim 1, wherein the zwitterionic moieties are present in both the superficial domain, and the subsurface interpenetrated network domain with a progressively diminishing density of the primary interpenetrated network at increasing depths.

12. The medical device of claim 1, wherein the surface region is anti-fouling.

13. The medical device of claim 1, wherein the surface region further comprises non-leachable biocides or leachable antibiotics.

14. A method of manufacturing an indwelling medical device, comprising:
swelling a medical device in a first solution to facilitate penetration of precursors into the medical device; and
submerging the medical device in a second solution to polymerize an interpenetrated polymer network;
wherein
the first solution comprises hydrophobic monomers, radical initiator, and a solvent,
the second solution comprises hydrophilic monomers, catalyst and a solvent, and
the solutions are configured to form a core region comprising more than 64% of the volume of the indwelling medical device, and a surface region comprising less than 36% of the volume of the indwelling medical device, the surface region comprising an interpenetrated polymer network comprising a primary polymer network and a secondary polymer network, wherein the interpenetrated polymer network has a thickness of more than 100 nm.

15. The method of claim 14, wherein the hydrophobic monomers are crosslinkers and comonomers that are configured to form a part of the secondary polymer network.

16. The method of claim 14, wherein the hydrophilic monomers are configured to form the majority of the secondary polymer network.

17. The method of claim 14, wherein the solutions are configured to form a surface region comprising a superficial domain and a subsurface interpenetrated network domain, wherein the superficial domain is the outermost layer of the surface region, wherein the subsurface interpenetrated network domain lies beneath the superficial domain.

18. The method of claim 14, wherein the solutions are configured to form a superficial domain that is highly hydrophilic and possess a large percentage of zwitterionic moieties, and a subsurface that comprises predominately of the primary polymer network.

19. The method of claim 14, wherein the solutions are configured to form a surface region wherein zwitterionic moieties are present in both the superficial domain and the subsurface interpenetrated network domain with a progressively diminishing density of the interpenetrated network at increasing depths.

20. The method of claim 14, further comprising submerging the medical device in a solution containing antibiotics or biocides to load or impregnate the medical device with non-leachable biocides or leachable antibiotics.
